(19) United States
(12) Patent Application Publication
(10) Pub. No.: US 2012/0239145 A1
(43) Pub. Date: Sep. 20, 2012

(54) STRONG POROUS TRANS-TENDON REPAIR DEVICE WITH SUTURE TAILS

(76) Inventors: Dale R. Peterson, La Jolla, CA (US); Peter Popper, Wilmington, DE (US); Justin C. Anderson, Minnetonka, MN (US); Kevin Ohashi, Jamaica Plain, MA (US); Ralph Mattern, San Diego, CA (US); Jamal Rushdy, Eden Prairie, MN (US)

(21) Appl. No.: 13/230,623

(22) Filed: Sep. 12, 2011

Related U.S. Application Data
(63) Continuation-in-part of application No. PCT/US2010/027158, filed on Mar. 12, 2010.
(60) Provisional application No. 61/160,129, filed on Mar. 13, 2009.

(30) Foreign Application Priority Data
Aug. 20, 2009 (FR) 0955715

(51) Int. Cl. A61F 2/08

(52) U.S. Cl. 623/13.14

(57) ABSTRACT

This reinforcing band (1) for restoring a soft tissue, such as a tendon or a ligament, comprises a textile component with at least one free edge (2, 4). In addition, the or each free edge is folded back into an inner volume of the textile component in such a way as to define an attachment edge (6, 8) suitable for attachment to the soft tissue.

### CROSS-REFERENCE TO RELATED APPLICATION

The present application is a continuation-in-part of Patent Cooperation Treaty Application No. PCT/US2010/027158, filed Mar. 12, 2010 and published as WO 2010/105171 A1 on Sep. 16, 2010, which claims the benefit of U.S. Provisional Patent Application Ser. No. 61/160,129, filed on Mar. 13, 2009, and which also claims the priority benefit of French Patent Application No. FR0955715, filed on Aug. 20, 2009, all of which are incorporated by reference herein in their entireties for all purposes.

### TECHNICAL FIELD

Embodiments of the present invention relate generally to reinforcing textile bands for repair of soft tissue, and more particularly to the connection of such reinforcing textile bands to suture.

### BACKGROUND

When a soft tissue of a human, such as a tendon or a ligament, is damaged at the point at which it attaches to the bone, the attachment may be restored in various ways, such as with sutures and screws. The corresponding surgical procedure is often long and delicate to implement. Despite the care exercised by the surgeon during the procedure, the risks of separation of the suture, so much more at the level of the soft tissue than the bone, are often elevated because, very often, the means used to join the tissue with the bone weakens the soft tissue and/or the bone. This fact is even more critical in the context of restoring the attachment of tendons and ligaments of a rotator cuff to a shoulder bone of a human being, due to the strong mechanical forces attained during shoulder movements.

Arthroscopic repair of rotator cuff tendons is difficult and repairs often fail when the sutures pull through the tendon, or, less often, through the bone tunnel. Use of a patch to reinforce the repairs generally adds considerable time and effort to the surgical procedure. Patch repairs are typically done in open or “mini-open” surgery, and a small number of surgeons who do patch repairs arthroscopically often add forty to ninety minutes to each surgery.

Although the threading of transferred tendons through rotator cuff or Achilles tendon tissue has been practiced by surgeons, such procedures have their own limitations.

### SUMMARY

The present invention relates to a reinforcing band for restoring a soft tissue, such as a tendon or a ligament, and to a method for producing such a band.

Following damage to or tearing of soft fibrous tissues, such as a ligament or a tendon, it is known to use a textile reinforcement. This textile component makes it possible to reinforce or restore the tissue. It is often beneficial for these reinforcements to have considerable resistance to tearing, in such a way as to remain joined to the tissue in the event of future stresses. To achieve this, such reinforcements can comprise rings or washers that are designed to permit fixing to the tissue by screws, for example. However, the design of these reinforcements is predefined and, consequently, does not allow the size of the reinforcement to be adapted, which size may depend on the diagnosis made by the surgeon.

Some embodiments of the present invention make available a reinforcing band for restoring soft tissues, with such band having substantial resistance to tearing and at the same time affording the surgeon flexibility of use during the surgery.

To this end, an embodiment of the invention is a reinforcing band for restoring a soft tissue, such as a tendon or a ligament, comprising a textile component with at least one free edge. In addition, the free edge or each free edge is folded back into an inner volume of the textile component in such a way as to define an attachment edge suitable for attachment to the soft tissue.

By virtue of the reinforcing band according to an embodiment of the invention, the surgeon is provided with a robust and reliable solution and is easily able to adapt the size before or during the surgery. This adjustment in size can be effected over a large extent, and in any area of the band, without the latter having to be cut.

A reinforcing band according to an embodiment of the present the invention may include one or more of the following possible technical features or characteristics:
it comprises a second free edge opposite the first free edge, the second free edge being folded back on the textile component in such a way as to define a second attachment edge suitable for attachment to the tissue, and the distance between the two free edges being substantially less than the distance between the two attachment edges;
that part of the textile component which is defined between the free edge and the corresponding attachment edge forms a cuff;
the textile component has a flexibility such as to allow the cuff or each cuff to at least partly conform to an inner wall of the component;
the textile component is elongate and hollow, in such a way that the free edge or each free edge is folded back inside the textile component;
the textile component is made of gauze;
the textile component comprises two strips joined in a region close to the attachment edges;
the textile component forms a loop;
the textile component is composed of biodegradable polymers, for example poly-4-hydroxybutyrate.

A method for producing a reinforcing band suitable for restoring a soft tissue, such as a tendon or a ligament, according to an embodiment of the present invention may include the following steps:
cutting at least one edge of a textile component of the reinforcing band, and
folding the free edge thus formed back on the textile component, in such a way as to form an attachment edge suitable for attachment to the soft tissue.

A method for restoring a soft tissue, such as a ligament or a tendon, according to an embodiment of the present invention may include the following steps:
cutting at least one edge of a textile component of a reinforcing band,
folding the free edge or each free edge thus formed back on the textile component, in such a way as to define an attachment edge,
joining the attachment edge to the soft tissue.

The step in which the free edge or each free edge is folded back may advantageously be carried out during the surgical intervention.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a reinforcing band according to a first embodiment of the present invention;

FIG. 2 is a schematic view of a placement of the reinforcing band from FIG. 1 in the area of a tendon;

FIG. 3 is a schematic view of the reinforcing band after it has been joined to the tendon;

FIGS. 4 and 5 are schematic views of a reinforcing band according to a second embodiment of the invention,

FIGS. 6 and 7 are schematic views of a reinforcing band according to a third embodiment of the invention; and

FIGS. 8 and 9 are schematic views of a reinforcing band according to a fourth embodiment of the invention.

FIG. 10 illustrates a reinforcing band with suture sewn to each end, according to embodiments of the present invention.

FIG. 11 illustrates a reinforcing band with suture tied to each end and extending from a side of the knot, according to embodiments of the present invention.

FIG. 12 illustrates a reinforcing band with suture tied to each end and extending from a center of the knot, according to embodiments of the present invention.

FIG. 13 illustrates a partial perspective view of a woven textile structure for a reinforcing band taken along circle XIII of FIG. 12, according to embodiments of the present invention.

FIG. 14 illustrates a partial cross-sectional view of an attachment of a suture to a reinforcing band, similar to the embodiment of FIG. 12, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

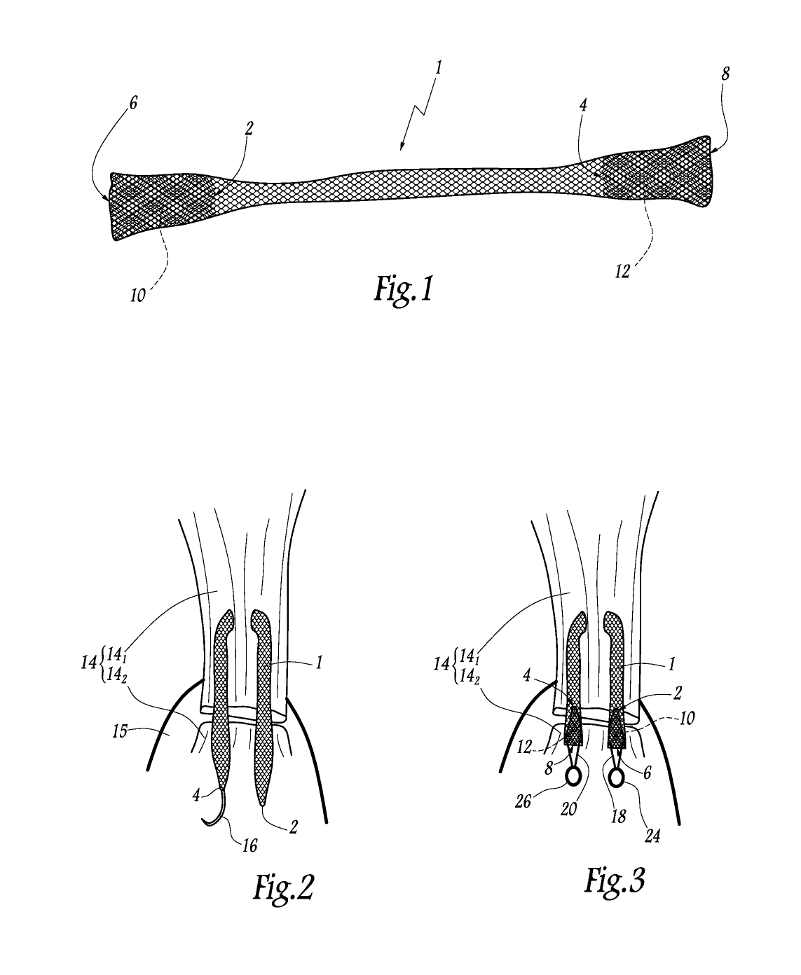

FIG. 1 illustrates a reinforcing band 1 that is elongate and hollow, thereby defining an inner volume. The band 1 comprises a textile component having two cut edges or free edges 2 and 4 arranged opposite each other. These free edges are folded back on the textile component towards the inside of the band in the direction of the inner volume. Two attachment edges 6 and 8 are thus defined which are arranged at each of the ends of the band 1 opposite each other. The attachment edge 6 is formed near the free edge 2, while the attachment edge 8 is formed near the free edge 4. The parts of the textile component that are defined between the edges 2 and 6, on the one hand, and between the edges 4 and 8, on the other hand, form inner cuffs 10 and 12, respectively. The flexibility of the textile component allows each of the cuffs to conform to the inner wall of the component. With this configuration, the distance between the free edges 2 and 4 is therefore less than the distance between the attachment edges 6 and 8.

The principal length of the reinforcing band 1 can thus be easily adjusted since, in order to do this, it suffices to modify the longitudinal dimension of each of the cuffs 10 and 12. The further the free edge 2 slides inside the hollow body of the band 1, the more the principal dimension of the band decreases. The same applies if the free edge 4 slides inside the hollow body. It should be noted that the dimension of each of the inner cuffs 10 and 12 can be different.

The zone of the textile component near the free edges 2 and 4 loses mechanical strength. The cutting of the textile component generates protruding threads, which mechanically weaken the structure at this location. Such a zone, fixed to a tissue, no longer has sufficient resistance to tearing. However, the attachment edges have not been cut in the textile component. By joining these edges 6 and 8 to the tissue, the resistance to tearing is increased.

Moreover, by increasing the longitudinal dimension of the cuffs 10, 12, a reinforcing band of greater mechanical strength is obtained, since it is less exposed to possible unravelling near the free edges. This is because the distance separating the free edges from the attachment edges is substantial.

In one embodiment, the textile component is composed of threads that are braided in two distinct axes. Alternatively, the threads of the textile component can define three distinct axes. Each of the threads can be either a monofilament or a multifilament and can be twisted helically in relation to the central axis of the thread, according to some embodiments of the present invention.

The textile component can be made of gauze. The mechanical strength and rigidity are thus improved.

The surgeon may choose to produce the reinforcing band 1 shown in FIG. 1 before starting the intervention on the tendon. He may also decide to produce it during the surgical intervention. To do this, he begins by cutting the textile component from a textile assembly in the area of the edges 2 and 4. He then folds the two edges into the inside of the textile component in such a way as to form the cuffs 10 and 12. The practitioner introduces the free edges 2 and 4 into the inside of the band 1 to a greater or lesser depth, depending on the desired size of the textile component. Alternatively, the steps mentioned above may be taken inside the human body, during arthroscopy, with just one hand manipulating a single instrument, such as a probe or a grip. It will be appreciated therefore that the cuffs 10 and 12 are not prefabricated and instead are produced during the operation; this band has no such cuffs prior to the surgical intervention for implanting the band.

FIGS. 2 and 3 illustrate the placement of the reinforcing band 1 with the aim of restoring a joint following damage to a tendon 14 fixed on the bone 15. A needle 16, shown in FIG. 2, is placed on the end of the band 1 in order to facilitate the introduction of the latter through the part 14₁ of the tendon 14. The surgeon begins by passing the needle through the part 14₁, then folds the free edges 2 and 4 back on the textile component in such a way that the band 1 has the desired length. The cuffs 10 and 12 thus partly conform to the inner wall of the textile component. Sutures 18 and 20 are then connected to the attachment edges 6 and 8 thus formed, respectively, and sutures 18 and 20 are themselves connected to the bone 15 by respective anchoring rings 24 and 26. The sutures are connected to the band using the existing openings in the braided structure of the band.

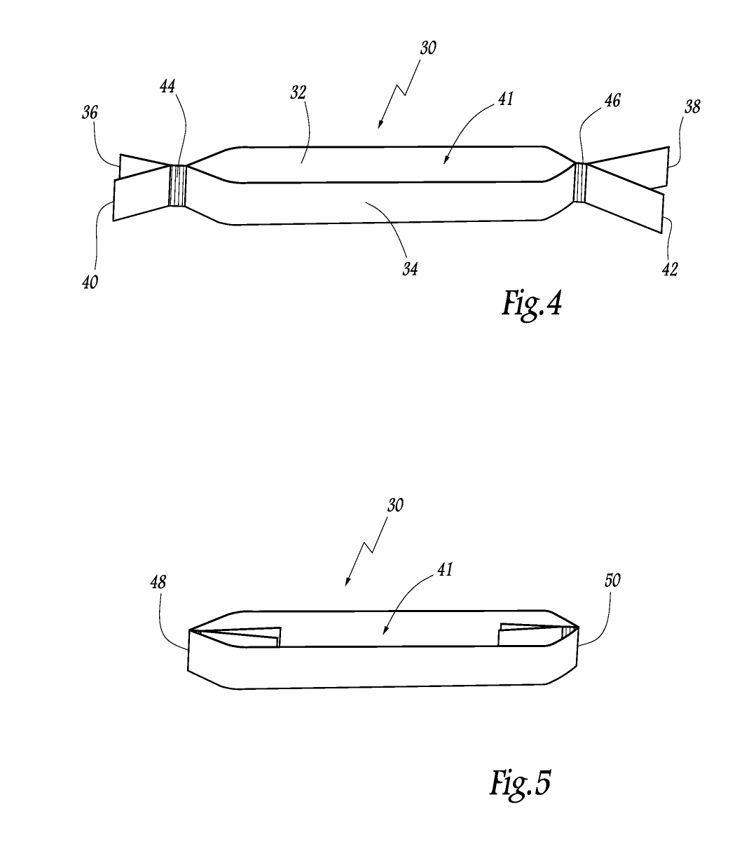

FIG. 4 illustrates a reinforcing band 30 comprising two strips 32 and 34 of textile component. The strip 32 comprises two opposite cut edges or free edges 36 and 38, while the strip 34 comprises two opposite cut edges or free edges 40 and 42. The two strips are joined to each other at the areas 44 and 46 by weaving, by suturing or by adhesive bonding, thereby defining an inner volume 41. By folding the free edges 38 and 42, on the one hand, and the free edges 36 and 40, on the other hand, back in the direction of the inner volume, the reinforcing band 30 illustrated in FIG. 5 is obtained. This band comprises two attachment edges 48 and 50 suitable for joining to a tissue.

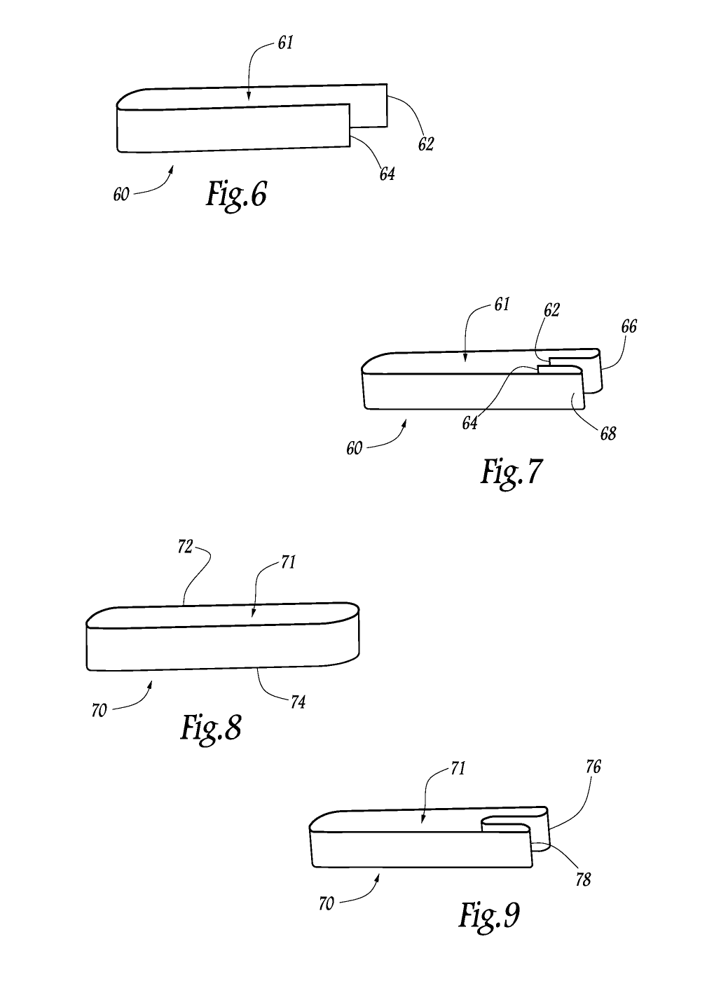

FIG. 6 illustrates a woven or braided reinforcing band 60 with two free edges 62 and 64. The band 60 defines an inner volume 61. As is shown in FIG. 7, by folding the two free edges back in the direction of the inner volume 61, then connecting them by suturing, by weaving, by interlacing or by adhesive bonding, the reinforcing band 60 is then in the form of a loop. The loop thus comprises two attachment edges 66 and 68 suitable for attachment to the soft tissue.

FIG. 8 illustrates a reinforcing band 70 in the form of a loop defining an inner volume 71. Each of the perimeters 72 and 74 corresponds to a cut edge or free edge. By folding part of the band 70 back into the inside of the loop, as is shown in FIG. 9, the longitudinal dimension of the reinforcing band is reduced. The loop thus defines two attachment edges 76 and 78 suitable for attachment to the soft tissue. To overcome the fact that threads protrude from the free edges 72 and 74 after cutting, provision can be made for each of the edges to be folded back on itself all around the perimeter into the inside of the loop. In other words, a lining is produced whose transverse dimension, that is to say width, is substantially less than the transverse dimension of the band 70. The textile component of the reinforcing band can be woven or braided.

The reinforcing bands 61 and 71 shown in FIGS. 7 and 9 can be produced before or during the surgical intervention.

In embodiments of the invention, the entire perimeter of the reinforcing band has no threads protruding from it. The mechanical strength of the band is all the greater as a result.

The textile component of each of the reinforcing bands may be composed of biodegradable polymers, for example poly-4-hydroxybutyrate.

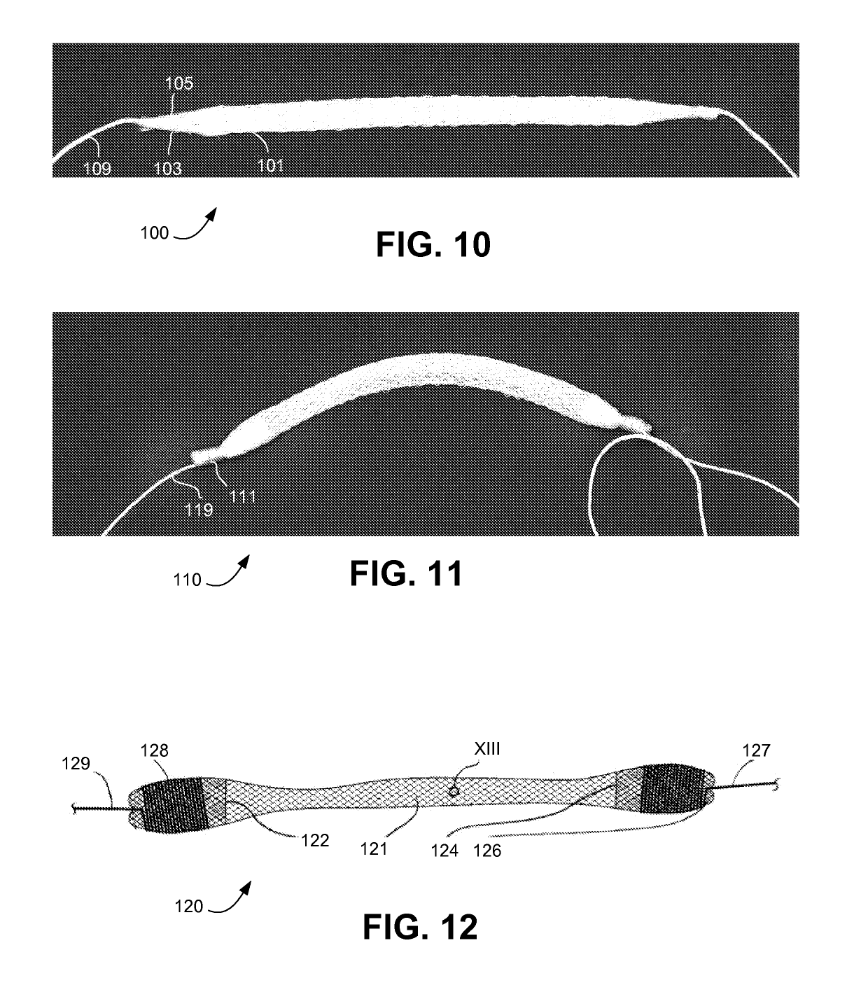

FIG. 10 illustrates a reinforcing band 100 with suture attached to each end, according to embodiments of the present invention. Reinforcing band 100 may be similar to reinforcing band 1, with the addition of suture on one or both ends, according to embodiments of the present invention. The suture 109 is sewn to the body 101 of the reinforcing band 100 at each end, and each end is then cut and/or shaped with a heat cut process, for example using a hot blade to form the edges 103, 105, which also fuses the body 101 and suture 109 together and creates a streamlined end which more smoothly transitions from body 101 to suture 109, according to embodiments of the present invention.

FIG. 11 illustrates a reinforcing band 110 with an alternative suture attachment to each end, according to embodiments of the present invention. Reinforcing band 110 may be similar to band 1, with the addition of suture on one or both ends. The suture 119 is tied around the end of the reinforcing band 110 with a classic Snell knot 111, and the suture 119 extends from a side circumference of the knot 111 or braid, rather than from the center of the braid, according to embodiments of the present invention.

FIG. 12 illustrates a reinforcing band 120 with an alternative suture attachment to each end, according to embodiments of the present invention. FIG. 12 illustrates the same body 121, which may also include free edges 122, 124 folded inwardly on themselves, and the same knot 128 as in FIG. 11, except the suture 127, 129 extends from the knot and/or from the braided body 121 at a center 126 of the end of the band 120, according to embodiments of the present invention. This arrangement may further deter snagging of the braided body 121 on the tissue, according to embodiments of the present invention.

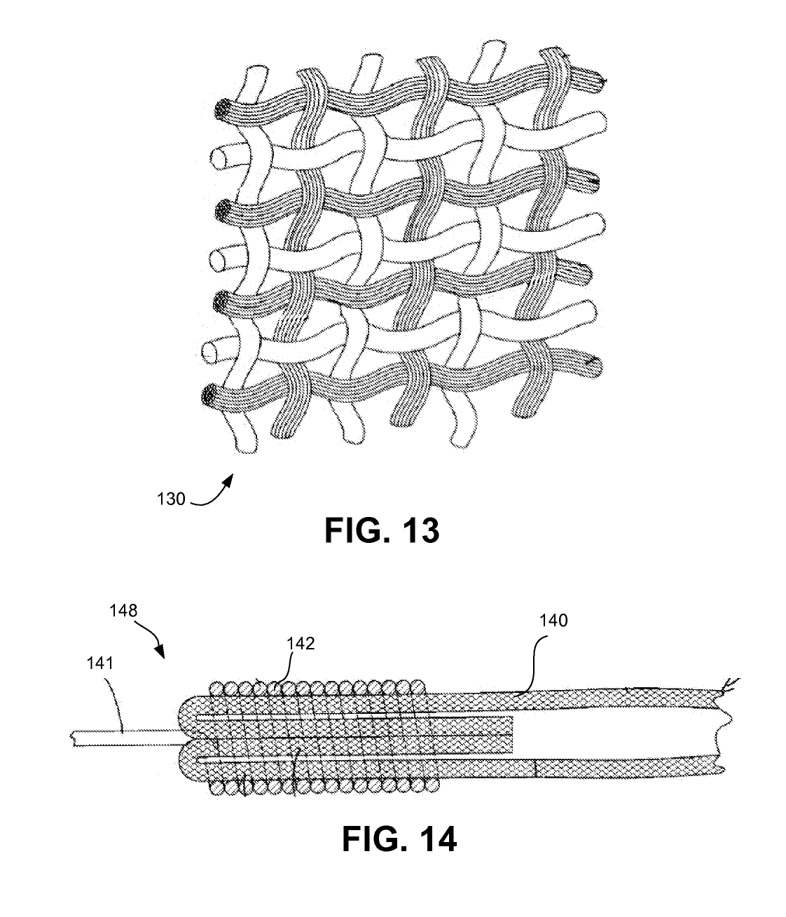

FIG. 13 illustrates a partial perspective view of a woven textile structure 130 for a reinforcing band, taken along circle XIII of FIG. 12, according to embodiments of the present invention. FIG. 14 illustrates a partial cross-sectional view of an attachment of a suture 141 to a reinforcing band 140, according to embodiments of the present invention. According to some embodiments of the present invention, the reinforcing band 140 is a braided and/or woven textile structure similar to bands 1 or 30 which is folded onto itself. FIG. 14 illustrates a suture 141 wrapped around the end 148 of the reinforcing band 140 to create one or more coils 142 or loops. The suture 141 may be tied in a knot, and/or threaded through the reinforcing band 140 and its ends, and/or stitched to the reinforcing band 140 at end 148, according to embodiments of the present invention. Attaching a suture 141 to one or more ends of the reinforcing band 140 permits the reinforcing band 140 to be used for the primary contact with the soft tissue (e.g. placement around or through the soft tissue such as a ligament or tendon), while also permitting the reinforcing band 140 to be maneuvered, threaded, and anchored with hardware that works best with sutures rather than textile bands. For example, the suture 141 ends can be threaded through a knotless suture anchor system, which might otherwise be to narrow or thin to accept the textile band 140 itself. Also, the textile band 140 may be more expensive to manufacture than the suture 141, so the surgeon may use only enough textile band 140 to contact the soft tissue, and bridge the rest of the distance to an anchoring site with suture. FIG. 14 illustrates one way to attach the suture 141 to the reinforcing band 140, according to embodiments of the present invention.

Some embodiments of the present invention may include one or more of the following characteristics and/or features:

A strong textile structure suitable (e.g. reinforcing bands 100, 110, 120, 140) for passing through a tendon which maintains porosity under tension.

A textile device for tendon repair comprised of overlapping monofilament fibers to maintain porosity under load (see, e.g., FIG. 13).

The textile device of paragraphs [0061] or [0062] made of resorbable biocompatible polymers.

The textile device of paragraphs [0061] or [0062] coupled to high strength suture for arthroscopic delivery, as illustrated in FIGS. 10-12 and 14.

A suture to textile structure coupling stronger than 300N.

A suture coupled to a reinforcing band so that load is transmitted to the porous reinforcing band and to ingrowing tissue so as to encourage remodeling of the tissue.

The textile device of paragraphs [0061] or [0062] (e.g. device 140) sewn to a suture (e.g. suture 141).

The textile device of paragraphs [0061] or [0062] (e.g. device 140) tied to a suture (e.g. suture 141).

The textile device of paragraph [0068], tied to a suture with a Snell knot.

The textile device of paragraphs [0061] or [0062] with tails of suture suitable for tensioning with a knotless anchoring system, for example the systems described in U.S. Pat. No. 7,938,847, issued on May 10, 2011, which is incorporated by reference herein in its entirety.

A textile reinforcing band stronger and/or stiffer than size 0 Fiberwire or suture tape.

A textile reinforcing band having a flat weave or braid structure.

A textile reinforcing band that is resorbable, for example made of Artelon, hydroxybutyrate, lactide, and/or glycolide, or permanent, for example made of polyester, nylon, PEEK, silk, polyacetal, polyurethane, polyaramide, and the like, or of a fiber construction.

A reinforcement method involving weaving a textile reinforcing band through a tendon with multiple passes.

A braided textile reinforcement structure for tissue ingrowth having both monofilament and multifilament yarns cut and fused at each end with heat or ultrasonic welding.

A braided textile reinforcement structure with both ends tucked into the core of the hollow braid to provide a strong stable end for attachment.

A medical textile device braided with 18 to 128 carriers of monofilament fiber and 18 to 128 carriers of multifilament yarn. The monofilament may be 80 to 500 microns in diameter and the multifilament yarns may be 60 to 1000 denier, according to embodiments of the present invention.

A medical textile device braided with 48 to 128 carriers of monofilament fiber and 48 to 128 carriers of multifilament yarn. The monofilament may be 100 to 300 microns in diameter and the multifilament yarns may be 100 to 350 denier, according to embodiments of the present invention.

Addition of biologics to the device, for example PRP, bone marrow aspirate, growth factors, peptides, collagen or other ECM molecules, attachment molecules, cytokines, signaling molecules, chemoattractants, stem or progenitor cells, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A reinforcing band for restoring a soft tissue, such as a tendon or ligament, comprising:
a textile component with at least one free edge, wherein the at least one free edge is folded back into an inner volume of the textile component; and
a suture coupled to the textile component at a location at which the at least one free edge is folded back into the inner volume of the textile component.

2. The reinforcing band of claim 1, wherein the suture is tied to the textile component at the location.

3. The reinforcing band of claim 2, wherein the suture is tied to the textile component at the location with a Snell knot.

4. The reinforcing band of claim 1, wherein the suture is stitched to the textile component at the location.

5. The reinforcing band of claim 1, wherein the coupling of the suture and the textile component is capable of withstanding a tension force of at least three hundred Newtons.

6. The reinforcing band of claim 1, wherein the textile component comprises a flat weave.

7. The reinforcing band of claim 1, wherein the textile component comprises a braid.

8. The reinforcing band of claim 1, wherein the textile component comprises monofilaments and multifilament yarns fused at each end.

9. The reinforcing band of claim 1, wherein the textile component is braided with eighteen to one hundred twenty-eight carriers of monofilament fiber and eighteen to one hundred twenty-eight carriers of multifilament yarn.

10. The reinforcing band of claim 9, wherein the monofilament fiber is eighty to five hundred microns in diameter, and wherein the multifilament yarn is sixty to one thousand denier.

11. The reinforcing band of claim 9, wherein the textile component is braided with forty-eight to one hundred twenty-eight carriers of monofilament fiber and forty-eight to one hundred twenty-eight carriers of multifilament yarn.

12. The reinforcing band of claim 11, wherein the monofilament fiber is one hundred to three hundred microns in diameter, and wherein the multifilament yarn is one hundred to three hundred fifty denier.

13. The reinforcing band of claim 1, wherein the textile component is resorbable.

14. A method for making a reinforcing band for restoring a soft tissue, such as a tendon or ligament, the method comprising:
forming a textile component with at least one free edge;
folding the at least one free edge back into an inner volume of the textile component; and
coupling a suture to the textile component at a location at which the at least one free edge is folded back into the inner volume of the textile component.

15. The method of claim 14, wherein coupling the suture to the textile component comprises tying the suture to the textile component at the location.

16. The method of claim 14, wherein tying the suture to the textile component comprises tying the suture to the textile component with a Snell knot.

17. The method of claim 14, wherein coupling the suture to the textile component comprises stitching the suture to the textile component at the location.

18. The method of claim 14, wherein forming the textile component further comprises braiding monofilament fibers and multifilament yarns to form a braided structure, and cutting and fusing each end of the braided structure to form the textile component.

19. The method of claim 14, further comprising adding biologic agents to the textile component.

20. The method of claim 19, wherein the biologic agents are one or more agents selected from the group consisting of platelet-rich plasma, bone marrow aspirate, growth factors, peptides, collagen or other extracellular matrix molecules, attachment molecules, cytokines, signaling molecules, chemoattractants, stem cells, and progenitor cells.
